(19) United States
(12) Patent Application Publication
(10) Pub. No.: US 2016/0199392 A1
(43) Pub. Date: Jul. 14, 2016

(54) METHOD FOR INDIVIDUALIZED DRUG THERAPY

(71) Applicant: Autotelic LLC, City of Industry, CA (US)

(72) Inventor: Vuong Trieu, Agoura Hills, CA (US)

(21) Appl. No.: 14/595,823

(22) Filed: Jan. 13, 2015

(51) Int. Cl. A61K 31/60; A61K 45/06; A61K 31/415

(52) U.S. Cl. CPC A61K 31/60; A61K 31/415; A61K 45/06

(57) ABSTRACT

Methods for individualized therapy of arthritic pain using a non-steroidal anti-inflammatory drug (NSAID). Said methods comprise basing NSAID dose on each patient's pharmacokinetic and pharmacodynamic response to said NSAID.

### BACKGROUND OF THE INVENTION

The metabolism of a particular drug, and as a result the blood concentration and the duration of action achieved by that drug, can vary significantly in a general population. (See Chun-Yu et al.: Pharmacogenomics of adverse drug reactions: Implementing personalized medicine, Human Molecular Genetics, 2012, 21, Review Issue 1, B58-B65). In the care of a patient suffering from a particular disease, a drug's efficacy against diseases is a fundamental issue. However, side effects or “adverse drug reactions” (“ADRs”) caused by a drug can profoundly impact the patient, thus requiring alterations in the treatment plan. (See Lazarou et al.: Incidence of adverse drug reactions in hospitalized patients: a meta-analysis of prospective studies. JAMA 1998, 279(15):1200-5). ADRs account for ˜7% of all hospitalizations and consistently rank as one of the most common causes of inpatient death in western countries. (See Pirmohamed & Park: Adverse drug reactions: back to the future. Br. J. Clin. Pharmacol., 2003, 55, 486-492; Wester et al.: Incidence of fatal adverse drug reactions: a population based study, Br. J. Clin. Pharmacol. 2007, 65:4, 573-579).

To guard against ADRs, administering the lowest dose of a drug to a patient that achieves the greatest efficacy of the drug is of paramount importance because 75%-80% of all ADRs are dose-related (i.e., the patient experiences a side effect because they are taking too high of a dose of a particular medication). (See Routlege et al.: Adverse drug reactions in elderly patients, Br J Clin Pharmacol 2004, 57:2 121-126; Lazarou et al.: Incidence of adverse drug reactions in hospitalized patients: a meta-analysis of prospective studies. JAMA 1998, 279 (15):1200-5.; Melmon, K L, Morrelli, H F, Hoffman, B B, Nierenberg, D W. Melmon and Morrelli's Clinical Pharmacology: Basic Principles in Therapeutics. (3rd Edition), New York: McGraw-Hill, Inc., 1993). Despite the risks from ADRs, finding the lowest effective dose is often not addressed by conventional prescribing regimens. Additionally, the variability in individual responses to a drug significantly complicates finding the lowest effective dose. The experiences of prior patients may not be relevant to a particular individual patient's regimen. Prescribers may be deterred by a complexity of finding the lowest effective dose, and as a result, because many patients are maintained on an effective dose rather than the lowest effective dose, inadequate patient responses to the drug and/or responses with significant ADRs may occur.

Although differences in age, gender, and size contribute to the heterogeneity in drug metabolism, patients that are the same age, gender, and size can experience markedly different responses to the same drug dosage. Other factors which can influence the likelihood of ADRs include, without limitation, the administration of multiple drugs, disease state, past history of ADRs, allergic reactions, and genetic factors effecting the absorption, distribution, chemical alteration, and excretion of the drug. The ADRs from the class of drugs known as “non-steroidal anti-inflammatory drugs” (“NSAIDS”) are well documented. (See, e.g., Dieppe et al.: Balancing benefits and harms: the example of non-steroidal anti-inflammatory drugs, BMJ 2004, 329, 31-34; McGettigan & Henry: Cardiovascular Risk with Non-Steroidal Anti-Inflammatory Drugs: Systematic Review of Population-Based Controlled Observational Studies, PLoS Med 2011, 8(9): e1001098. doi:10.1371/journal.pmed.1001098; Aagaard & Hansen: Information about ADRs explored by pharmacovigilance approaches: a qualitative review of studies on antibiotics, SSRIs and NSAIDs, BMC Clinical Pharmacology 2009, 9:4; Süleyman: Anti-inflammatory and side effects of cyclooxygenase inhibitors, Pharm. Reports 2007, 59, 247-58). Further, although newer NSAIDS have somewhat reduced the risk for gastrointestinal bleeding, ulceration, and perforation, they still present risks to patients such as kidney failure, hepatic dysfunction, and cardiovascular events (e.g., stroke, hypertension, congestive heart failure) (See Celebrex® package insert; see also Bing, et al.: Cyclooxygenase-2 inhibitors: is there an association with coronary or renal events?, Current Atherosclerosis Reports 2003; 5:114-7.)

The danger of NSAID ADRs is particularly important in the elderly (i.e., age>65) where drug metabolism is quite heterogeneous. (See Singh et al: Gastrointestinal Drug Interactions Affecting the Elderly, Clin. Geriatr. Med 2014, 30:1-15). As individuals age, they begin to experience diminished organ function, suffer from various diseases, and often take drugs that can interact resulting in an increased susceptibility to environmental and physical stressors (e.g., medications). As a result, seniors are generally more susceptible to the harmful side effects of NSAIDs, and yet generally receive the same dosing regimens as larger, younger individuals. (See McMillan & Hubbard: Frailty in older inpatients: what physicians need to know, Q. J. Med. 2012; 105:1059-1065.; Smucker & Kontak: Adverse drug reactions causing hospital admission in an elderly population: experience with a decision algorithm, Journal of the American Board of Family Practice 1990, 3(2):105-9; Montamat et al.: Management of drug therapy in the elderly, New England Journal of Medicine 1989, 321(5):303-9; and Recchia & Shear: Organization And Function Of An Adverse Drug Reaction Clinic. Journal Of Clinical Psychiatry 1994, 34:68-79).

Celecoxib (sold under the brand name “Celebrex®”) is a NSAID that has been approved for the treatment of arthritis for over 15 years. In vitro assays demonstrate that celecoxib is a potent inhibitor of prostaglandin synthesis with most of its activity resulting from its inhibition of COX-2. (See Süleyman et al.: Anti-inflammatory and side effects of cyclooxygenase inhibitors, Pharm. Reports 2007, 59, 247-58). Like other NSAIDs, celecoxib puts patients, in particular elderly patients, at risk for a number of serious ADRs. For example, the 1999 Celebrex® package insert warns that “the incidence of adverse experiences tended to be higher in elderly patients” (Celebrex® Package Insert. Searle & Co., 1999). Similarly, the 2003 Celebrex® package insert states that “there have been more spontaneous post-marketing reports of fatal gastrointestinal events and acute renal failure” regarding its use of in the elderly. The 2013 package insert indicates that “Celebrex® should be used with caution in [elderly] patients.” The Celebrex® package insert advises that for osteoarthritis and rheumatoid arthritis, the lowest effective dose of Celebrex® should be sought for each patient.

Even the cyclooxgenase inhibitary activity of celecoxib appears to be variable (See McAdam et al.: Systemic biosynthesis of prostacyclin by cyclooxygenase (COX)-2: the human pharmacology of a selective inhibitor of COX-2. PNAS. 1999; 96:272-7.) FIG. 1 is a scatterplot graph from McAdams et al. displaying the relationship between LPS-stimulated plasma PGE2 ex vivo, an index of COX-2 activity, and log plasma concentrations of celecoxib at 2, 4, 6, and 24 hours after dosing. PGE2 is expressed as a percentage of predosing values. A variable dose-response is evident. (P, 0.01 vs. placebo).

A study by Bensen, et al. indicates that higher doses of celecoxib (100 mg and 200 mg twice a day) are similarly efficacious, while patients receiving the 100 mg dose of celecoxib also reported fewer side effects than those taking a 200 mg dose. (Table 1). (Bensen, et al.: Treatment of Osteoarthritis With Celecoxib, a Cyclooxygenase-2 Inhibitor: A Randomized Controlled Trial, Mayo Clinic Proceedings 1999, 74(11):1095-1105)

TABLE 1
Effect of Treatment on Signs and Symptoms of Osteoarthritis at 12 Weeks

| | Placebo | 50 mg BID | 100 mg BID | 200 mg BID | Naproxen |
|---|---|---|---|---|---|
| Assessment | (n = 203) | (n = 203) | (n = 197) | (n = 202) | (n = 198) |
| Patient global | 24 | 27‡ | 35‡ | 36‡ | 29‡ |
| assessment | | | | | |
| condition improved | | | | | |
| (%)† | | | | | |
| Patient global | 9 | <1 | 1 | <1 | 2 |
| assessment | | | | | |
| condition | | | | | |
| worsened (%)† | | | | | |
| Physician's global | 21 | 30‡ | 36‡ | 32‡ | 33‡ |
| assessment | | | | | |
| condition improved | | | | | |
| (%)† | | | | | |
| Physician's global | 4 | 2 | 2 | <1 | 1 |
| assessment | | | | | |
| condition | | | | | |
| worsened (%)† | | | | | |
| Mean +/− SEM | −6.1 +/− 1.09 | −9.5 +/− 1.11‡ | −13.3 +/− 1.17‡// | −12.0 +/− 1.22‡ | −11.9 +/− 1.29‡ |
| WOMAC | | | | | |
| Osteoarthritis Index | | | | | |
| composite score§ | | | | | |
| Mean +/− SEM | −2.0 +/− 0.29 | −3.3 +/− 0.32‡ | −3.8 +/− 0.29‡# | −3.4 +/− 0.27‡ | −3.1 +/− 0.30‡ |
| Osteoarthritis | | | | | |
| Severity Index¶ | | | | | |
| Withdrawal due to | 39 | 30 | 20‡// | 24‡ | 26‡ |
| treatment failure | | | | | |
| (%) | | | | | |

*BID = twice a day;
WOMAC = Western Ontario and McMaster Universities
†Scale ranged from 1 (very good) to 5 (very poor). Patient improvement was defined as a reduction of at least 2 grades from baseline for grades 3 through 5 or a change in grade from 2 t1. Patient worsening was defined as an increase of at least 2 grades from baseline for grades 1 through 3, or a change in grade from 4 to 5.
KEY:
‡= P ≦ .05 vs placebo;
§= Scale ranged from 0 to 96 with negative change indicating improvement
//= P ≦ .05 vs celecoxib, 50 mg BID;
¶= Scale ranged from 0 to 24 with negative change indicating improvement;
#= P ≦ .05 vs naproxen

Despite these acknowledgements, the Celebrex® dosage prescribed for the treatment of osteoarthritis, is standardized (e.g., 50, 100, 200 or 400 mg dosage forms) regardless of the gender and age of the patient receiving celecoxib. Furthermore, studies have shown that, on average, elderly patients receiving 100 mg celecoxib doses over a six week period reported almost identical reductions in pain as compared to patients receiving 200 mg celecoxib doses over a six week period. The subjects receiving the 100 mg dose of celecoxib also reported fewer side effects for the duration of the study. The results indicate that patients may experience the same celecoxib efficacy at lower, safer doses. Accordingly, there is a need for improved methods of using NSAIDs in the therapy of arthritic pain.

The present invention addresses the wide variability in the responses individuals have to the same dosage of a drug by setting forth methods for individualizing drug therapy. The methods of the present invention individualize dosages a patient takes, thus maximizing the efficacy of one or more drugs at the lowest doses possible and at the point of care. The present invention can increase drug compliance, reduce costs, and most importantly improve the quality and safety of patient lives.

### BRIEF SUMMARY OF THE INVENTION

The invention provides methods for individualized therapy of arthritic pain using a non-steroidal anti-inflammatory drug (NSAID). The high patient to patient variability in response to a dose of any NSAID makes the mere clinical monitoring of patients an inadequate way to treat patients with this class of drugs. Even the measurement of “blood levels'” (i.e., the occasional measurement of the drug's concentration in the blood) is unlikely to lead to effective nontoxic regimens. Given the complexity of NSAIDs' dose response relationships, a more compressive set of metrics must employed in each patient. The methods claimed herein take advantage of a pharmacokinetic (“PK”) analysis for each patient. As such, the claimed methods go beyond the measurement of a single blood level at a single time point. Instead, the claimed methods make use of data from several time points and take advantage of the full scope of PK parameters. There is no known method of predicting individual PK for celecoxib due to the complexity of human pharmacokinetics. As such there is no natural law known that can explain human pharmacokinetics; or if there are, the multitude of potential determining factors make defining such law impractical. Therefore, the claimed method seeks to determined the individual PK directly.

The inventive methods disclosed herein comprise: administering to a first patient suffering from arthritic pain a first NSAID formulation; determining the NSAID's concentration in the first patient's blood at a plurality of time points after the first NSAID formulation was administered to the first patient; transforming the first patient's NSAID concentration/time data points in to one or more PK parameters; comparing the first patient's values for said PK parameters to a predetermined ranges of values for each PK parameter and if one or more of the first patient's PK parameters fall outside of a predetermined range, designing a new NSAID formulation, wherein the dose of said NSAID can be different from that of the first NSAID formulation. As such, the types and amounts of excipients can also differ from those of the first NSAID formulation, or both. The new NSAID formulation is then administered to the first patient and the steps used to determine the PK parameters are repeated until all the PK parameters used are within said predetermined ranges, and if pain control is adequate and toxicity is tolerable, maintaining the first patient on the NSAID formulation at frequency of administration that satisfied the comparison within predetermined ranges.

Related methods for individualizing therapy of arthritic pain are disclosed herein.

### BRIEF DESCRIPTION OF THE DRAWINGS

FIG. 1 is a scatterplot graph displaying the relationship between LPS-stimulated plasma PGE2 ex vivo, an index of COX-2 activity, and log plasma concentrations of celecoxib 2, 4, 6, and 24 hours after dosing. PGE2 is expressed as a percentage of predosing values. A steep but variable dose-response is evident. (P, 0.01 vs. placebo) (from McAdam et al. Systemic biosynthesis of prostacyclin by cyclooxygenase (COX)-2: the human pharmacology of a selective inhibitor of COX-2. PNAS. 1999; 96:272-7.)

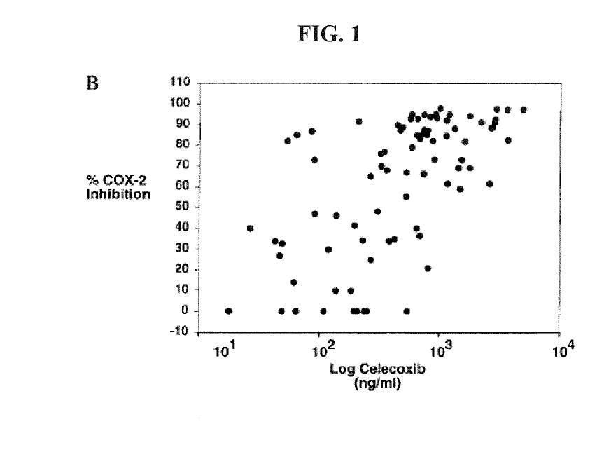

FIG. 2 depicts the pharmacokinetic parameters produced by different doses of celecoxib.

FIG. 3 displays the result of a meta-analysis of the one dose AUC from patients in different age groups.

### DETAILED DESCRIPTION OF THE INVENTION

As used herein the phrase “individualized therapy” refers to a specific treatment regimen for a patient comprising the administration of one or more drugs, which is the result of analyzing pharmacokinetic and/or pharmacodynamic parameters of the subject to maximize drug efficacy at about the lowest dosage of the drug(s) possible.

As used herein “Arthritic pain” refers to any pain arising anatomically from the joints and their adjacent bones and non-osseous tissues. Any arthritic pain can be treated by the invention including, without limitation, any pain resulting from an auto-immune, infectious, inflammatory, proliferative, regenerative or degenerative process so involving the joints of an animal or human patient. As such, suitable pain treatable with the current invention includes pain from rheumatoid or osteo arthritis.

As used herein, “formulation” or “a formulation” refers to a combination of active ingredients and excipients wherein each is present in a dosage form at fixed ratios to one another (i.e., fixed percentages of each ingredient in the dosage form.)

As used herein, “first NSAID formulation” is the NSAID formulation that is administered to the patient to begin the process by which PK parameters are determined and “new NSAID formulation” is the formulation designed based on the PK parameters produced by the first NSAID formulation. To evaluate the PK parameters produced by the new NSAID formulation becomes the first NSAID formulation and the process is repeated. The new NSAID formulation may be a second formulation.

As used herein, the phrases “second formulation” and “second formulation under a second drug regimen” refer to the dosage of one or more drugs an individual receives after performing certain steps of the claimed invention (e.g., after comparing an patient's pharmacokinetic parameters to a predetermined range of values). The second formulation under a second regimen, can be, e.g., the same dosage of a drug administered in the first regimen, a lower dose, or a higher dose. In addition, there can be changes in any non-NSAID components of any combination therapy.

As used herein the phrases “first formulation under a first regimen” and “first formulation” refer to the dosage of one or more drugs an individual initially receives prior to performing one or more steps of the claimed invention. The first formulation under a first regimen, can be, e.g., the standard 100 mg or 200 mg dosages of celecoxib prescribed to patients over 60 kg twice daily for osteoarthritis.

As used herein, the phrase “NSAID concentration/time data points” refers to the amount of NSAID in a unit of volume (e.g., 1 ml of blood from a subject) at a given point in time before or after administration of the NSAID.

As used herein, the phrase “transforming the patient's NSAID concentration/time data points” refers to the application of mathematical operations, formulas, theories, and/or principles (e.g., a formula for calculating half life or a formula for calculating AUC), to the NSAID concentrations/time data points of an individual to derive PK parameters.

As used herein, the phrase “predetermined range of values” refers to a known range of values (e.g., pharmacokinetic parameters) associated with desirable drug efficacy at lower risk doses of a drug (e.g., the Cₘₐₓ range corresponding to patients experiencing high drug efficacy at a low dose). The predetermined range of values may be derived from a statistical analysis of a population receiving variable and/or identical doses of one or more drugs. The predetermined range of values can readily be compared to a patient's values (e.g., pharmacokinetic values), associated with one or more drugs. In particular, the predetermined ranges are determined (i.e. derived) from other patients for whom there was improved pain levels without significant side effects.

As used herein, “Significant adverse drug reactions” refer to ADRs that the patient finds intolerable, impair physiologic functions, and/or put the patient at risk for immobility and/or death or combinations thereof.

As used herein “Significant side effects” refer to side effects that the patient finds intolerable, impair physiologic functions, and/or put the patient at risk for immobility and/or death or combinations thereof.

As used herein, “designing” refers to changes in the active agent's dose, formulation and/or regiment based on the patient data, using logic and the experience of one of ordinary skill in the art.

As used herein, “determining the level of efficacy” refers to the use of objective (e.g., pharmacokinetic) and subjective tests (e.g., pharmacodynamic), signs and symptoms to characterize, quantify or evaluate how well symptoms (e.g., pain) are controlled by the administration of the active ingredient (e.g., celecoxib).

As used herein, “determining the level of toxicity” refers to the use of objective and subjective tests, signs and symptoms to characterize, quantify or evaluate the significance of any side effects produced by the administration of the active ingredient.

As used herein, “pain control is adequate” refers to a level of pain the patient is willing to live with and does not significantly impair the patient's functioning in society or the patient's physiologic functions.

As used herein, “toxicity is acceptable” refers to the absence of significant side effects and a level of toxicity that the patient is willing to live with and does not significantly impair the patient's functioning in society or the patient's physiologic functions.

As used herein, a “COX-1 inhibitor” refers to a non-steroidal anti-inflammatory drug that is capable of directly targeting the COX-1 enzyme in a subject and inhibits at least some COX-1 activity, e.g., aspirin.

As used herein, a “COX-2 inhibitor” refers to a non-steroidal anti-inflammatory drug that is capable of directly targeting the COX-2 enzyme in a subject and inhibits at least some COX-1 activity, e.g., celecoxib. As used herein, a “mixed COX-1 and COX-2 inhibitor” refers to a non-steroidal anti-inflammatory drug that is capable of directly targeting both the COX-1 and COX-2 enzymes in a subject and inhibits at least some COX-1 and COX-2 activity, e.g., ibuprofen.

As used here a mixed COX-2 inhibitor can also include a COX-2 inhibitor such as celecoxib combined with one or more other therapeutic drugs for chronic diseases such as arthritis, diabetes, hypertension, hypercholesterolemia, and dementia. Suitable therapeutic drugs may include, but are not limited to: aspirin, diclofenac (Cataflam, Voltaren), diflusnisal (Dolobid), etodolac (Lodine), fenoprofen (Nalfon), flubiprofen (Ansaid), ibuprofen (Motrin, Advil, Nuprin), indomethacin (Indocin), ketoprofen (Orudis), ketorolac (Toradol), meclofenamate, nabumetone (Relafen), naproxen (Naprosyn), oxaprozin (Daypro), phenylbutazone, piroxicam (Feldene), salicylate, sulindac (Clinoril), tolmetin (Tolectin), pregbalin, neurontin, beclomethasone, betamethasone, cortisone, dexamethasone, hydrocortisone, methylprednisolone, prednisolone, prednisone, triamcinolone, sulfonylureas, acetohexamide (Dymelor), chlorpropamide (Diabinese), glimepiride (Amaryl), glipizide (Glucotrol), glyburide (Micronase, DiaBeta), tolazamide (Tolinase), tolbutamide (Orinase), Biguanides, metformin (Glucophage), Alpha-glucosidase Inhibitors, acarbose (Precose), miglitol (Glyset), Thiazolidinedione Derivatives, pioglitazone (Actos), rosiglitazone (Avandia), troglitazone (Rezulin), enalapril, lisinopril perindopril, losartan, valsartan, diltiazem, nifedipine, amlodipine, diuretics, amiloride, frusemide, indapamide, atenolol, metoprolol, propanolol, alpha-blockers, doxazosin, prazosin, methyldopa, vasodilators, and hydralazine.

As used here the combination can also include a “fixed dose combination” (FDC) or simply dosing with multiple pills each of a single agent to achieve a desired effect.

The invention provides methods for individualized therapy of arthritic pain using a non-steroidal anti-inflammatory drug (NSAID), preferably celecoxib. In addition, the invention provides methods for predicting the outcome of the therapy arthritic pain with a composition comprising NSAID, preferably celecoxib. Further, the invention provides methods of using a NSAID, including aspirin, preferably celecoxib, in the manufacture of medicament for the treatment of arthritic pain.

The invention provides endpoints (i.e., an individualized drug therapy) based on the achievement of predetermined PK results, as well as the clinical condition of the patient.

This disclosure provides processes in accordance with invention comprising: administering a first formulation under a first regimen comprising a NSAID, preferably celecoxib, at a first dose to a patient suffering from arthritic pain; at a pre-specified time or times after administration of the first dose of the NSAID, measuring the NSAID's concentration in the patient's blood (e.g., by a point of care device, or by other suitable techniques known in the art) at a plurality of time points after administering the first formulation resulting in a set of NSAID concentration/time data points; transforming the patient's NSAID concentration/time data points in to one or more pharmacokinetic (PK) parameters; comparing the patient's PK parameters to a predetermined ranges of values for such PK parameters and if one or more of the PK parameters fall outside of the predetermined range, providing a second formulation under a second regimen wherein the second formulation has a different dose of NSAID, preferably celecoxib, from the first formulation, or different regimen. These steps will be repeated until the PK parameters are within a second predetermined range.

The invention further provides methods for individualized drug therapy of arthritic pain with a NSAID, preferably celecoxib, comprising: administering a NSAID at a first dose under a first regimen to a patient suffering from arthritic pain; determining the concentration of the NSAID at a plurality of pre-specified time points after administration of the first dose of the NSAID; using the concentration/time data, deriving one or more pharmacokinetic (PK) parameters exhibited by the NSAID when administered to said patient; determining the level of efficacy and toxicity produced by the first dose and regiment; using the level of efficacy and toxicity produced by said first dose to determine a new dose of the NSAID; administering the new dose of the NSAID to the patient; and repeating the forgoing steps until pain control is adequate, toxicity is acceptable, and PK parameters are stable. The patient will thereafter be maintained at the final dose (i.e., an individualized drug therapy) that produced the results. However, if the patient has recurrent pain or toxicity again, one measures the PK parameters and adjust dose to provide the PK results determined by the original trials of the NSAID.

Any suitable NSAID can be used in accordance with the invention, including without limitation, a COX-1-specific inhibitor, a COX-2-specific inhibitor, a mixed COX-1 and 2 inhibitor or a combination thereof. As such, the NSAID can be a salicylate, propionic acid derivative, acetic acid derivative, enolic acid (oxicam) derivative, anthranilic acid derivative (fenamatea) or combinations thereof. Accordingly, the NSAID can be, aspirin (acetylsalicylic acid), ibuprofen, naproxen, indomethacin, sulindac, piroxicam, clonixin, preferably celecoxib or a combination thereof. In addition, the invention can be used with combinations of NSAIDs and other analgesic drugs such as lidocaine, opiates, acetaminophen, tricylic antidepressants, anticonvulsants, carbamazepine, gabapentin, and pregabalin; other anti-inflammatory drugs such as steroids and immunosuppressants. Further, the invention can be used with combinations of NSAIDs and other therapies for arthritis, including but not limited to, methotrexate and gold-salts.

The NSAID can be administered in accordance with the invention via any suitable route including, without limiting, orally, rectally, by inhalation, trans-cutaneously, by injection, intra-venously or intra-arterially. The non-NSAID component of any combination therapy can be administered in accordance with the invention by any suitable route including, without limiting, orally, rectally, by inhalation, trans-cutaneously, by injection, intra-venously or intra-arterially. Any suitable regimen can be used in accordance with the invention to administer two or more drug components, including without limitation, simultaneously (within minutes of one another), substantially simultaneously (within an hour of one another) or at different times.

Other treatments for chronic diseases can be included such as treatments for diabetes, cardiovascular diseases, dementia, cholesterol, and hypertension. For example, a method of individualizing an NSAID drug therapy (e.g., a celecoxib therapy), may be practiced in conjunction with the administration of a prescribed cholesterol regulator, such as atorvastatin.

Any suitable PK parameter or parameters can be used in accordance with the invention, including without limiting concentration, concentration time course, peak concentration, and time after administration to peak concentration, terminal half-life, AUC, bioavailability, absorption, distribution, metabolism, excretion, biotransformation, or a combination thereof.

Any suitable pharmacodynamic parameter or parameters can be used in accordance with the invention, including without limiting the physiological changes of cells, tissues and ligaments of a patient, patient or physician reported pain level, the frequency of side effects, or a combination thereof.

Any suitable method for the assessment of pain known to those of ordinary skill in the art can be used in accordance with the invention, including, but not limited to, one-dimensional pain intensity scales, Wisconsin Brief Pain Questionnaire, Brief Pain Inventory, The McGill Pain Questionnaire and the short-form, McGill Pain Questionnaire (See Breivik et al.: Assessment of pain, British Journal of Anaesthesia 2008, 101 (1): 17-24).

The following examples further illustrate the invention but, of course, should not be construed as in any way limiting its scope.

### Example 1

The approved prescribing information for Celebrex® as listed on its package insert for US/EU/ROW instructs that a physician should use lowest effective dose for the shortest duration consistent with treatment goals for the individual patient. For four of the six approved indications the package insert includes a 100 mg BID regimen:

1) Osteoarthritis (OA): 200 mg QD or 100 mg BID

2) Rheumatoid Arthritis (RA): 100 mg BID or 200 mg BID

3) Juvenile Rheumatoid Arthritis (JRA): 50 mg BID in patients 10-25 kg. 100 mg BID in patients more than 25 kg

4) Ankylosing Spondylitis (AS): 200 mg once daily single dose or 100 mg BID.

5) Acute Pain (AP) and 5) Primary Dysmenorrhea (PD). 400 mg initially, followed by 200 mg dose if needed on first day. On subsequent days, 200 mg BID as needed

Unexpectedly, however, the inventor's analysis of the actual prescribing behavior using Evaluate Pharma/IMS database determined that the 200 mg is the predominant dose being prescribed by physicians by more than 10 to 1. These data are consistent with data from a MEPS survey (Table 2) and Medicaid survey (Table 3). In view of predominance of the 200 mg dosage form sales and the evidence that the 100 mg and 200 mg doses produce overlapping PK and pharmacodynamic results, it is questionable that the package insert's admonition that “the lowest dose of Celebrex® should be sought for each patient” is followed. Instead, the data indicates that it is likely that there are numerous patients a risk for ADRs because their celecoxib dose is higher than it needs to be (i.e., e.g., 200 mg BID rather than 100 mg BID).

TABLE 2
MEPS Survey Data

| Proprietary | | Strength | USA sales | USA sales |
|---|---|---|---|---|
| Name | Package description | (mg) | 2008 ($m) | 2011 ($m) |
| 1CELEBREX | 100 capsule in bottle (0025-1525- | 200 | 1,553 | 1,650 |
| | 31) | | | |
| 2CELEBREX | 500 capsule in 1 bottle (0025- | 200 | 323 | 132 |
| | 1525-51) | | | |
| 3CELEBREX | 100 capsule in 1 bottle (0025- | 100 | 65 | 142 |
| | 1520-31) | | | |
| 5CELEBREX | 500 capsule in 1 bottle (0025- | 100 | — | 17 |
| | 1520-51) | | | |
| 10CELEBREX | 100 blister pack in 1 carton (0025- | 100 | — | 7 |
| | 1520-34) >1 capsule in 1 blister | | | |
| | pack | | | |
| CELEBREX | 120 capsule in 1 bottle (63629-3021- | 200 | — | 2 |
| | 5) | | | |
| CELEBREX | 30 capsule in 1 bottle, plastic (67544- | 200 | — | 32 |
| | 204-30) | | | |
| TOTAL | | | 1,989 | 1,982 |

TABLE 3
Prescribing information derived from Medicaid

| | | Number | Number | Number | Number | Number | Number | Number |
|---|---|---|---|---|---|---|---|---|
| | Dose | of RXs | of RXs | of RXs | of RXs | of RXs | of RXs | of RXs |
| Name | (mg) | 2008 | 2009 | 2010 | 2011 | 2012 | 2013 | 2014 |
| CELEBREX | 50 | 293 | 485 | 856 | 1169 | 1167 | 1396 | 726 |
| CELEBREX | 100 | 33,436 | 36,023 | 43,755 | 47,524 | 35,399 | 34,178 | 16,628 |
| CELEBREX | 200 | 320,628 | 330,521 | 380,546 | 384,404 | 285,764 | 250,985 | 114,532 |
| CELEBREX | 400 | 1,383 | 1,636 | 3,116 | 3,476 | 2,565 | 2,240 | 1,093 |
| TOTAL | | 355,740 | 368,665 | 428,273 | 436,573 | 324,895 | 288,799 | 132,979 |

### Example 2

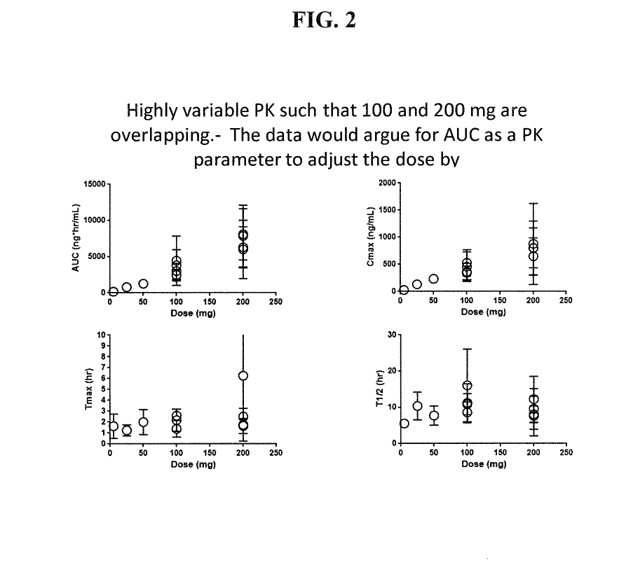

The combined plots of published pharmacokinetic data including those from the Summary basis for approval are shown in FIG. 2. The variability of Celebrex® pharmacokinetics were unexpectedly high. The PK results for the 200 mg dose shows a substantial overlap with that of the 100 mg dose. Accordingly, the dose proportionality may not be as is described by the package insert for Celebrex®. As a result of the failure to determine and pursue target PK ranges, in some instances patients receiving 100 mg patients may not get enough of the drug and the 200 mg patients may receive too much of the drug.

### Example 3

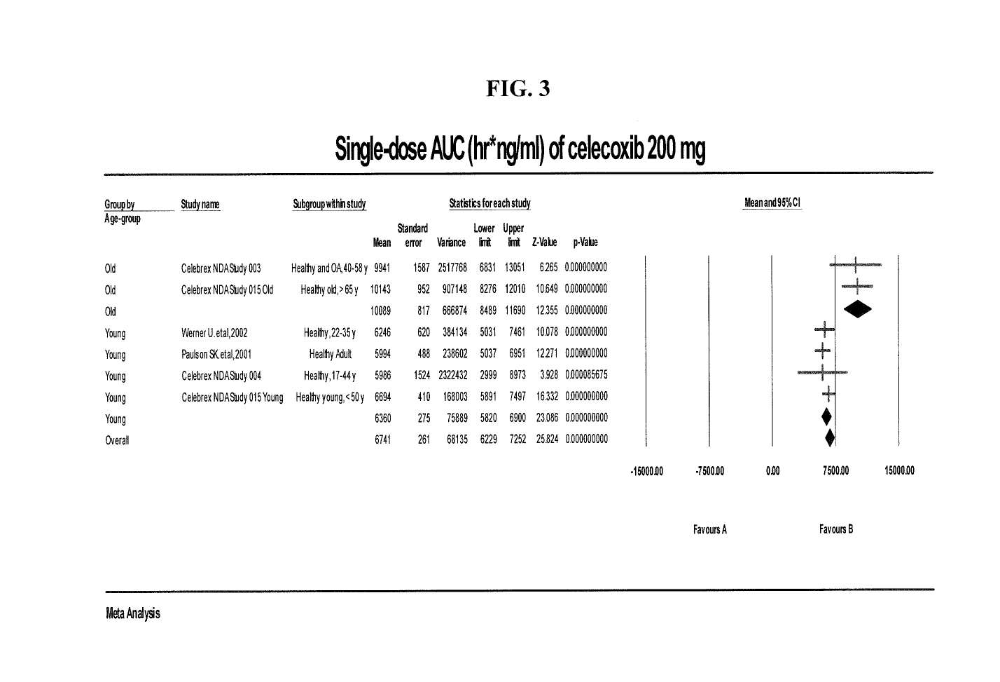

Applicant's meta analysis of the reported PK parameters in different populations demonstrates that the elderly show a higher variability than younger patients. For Example, when the applicant's meta analysis is presented in age-based subgroups, the elderly and younger patients demonstrate highly significant differences in the variability of AUC (FIG. 3). In other words, the most efficacious celecoxib dosage is highly individualized among the elderly. Unexpectedly, elderly here is defined as patients greater than >40 or >50, not the usually definition of elderly (age greater>65). Previously, there has been reported impaired PK with elderly and the package insert issued warning on impaired PK in elderly but did not suggest dose reduction. Our finding suggests that the issue is more substantial and more widespread and includes middle aged groups also.

That the variability of PK results within groups and the Cₘₐₓ and AUC overlap between the 100 mg and 200 mg groups indicates that correctly dosing elderly patients to maximize celecoxib efficacy at the lowest doses possible depends on many individualized, unpredictable variables. Based on the wide range of AUC values, some instances patients receiving 100 mg patients may not get enough of the drug and the 200 mg patients may receive too much of the drug (FIG. 2).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for individualized therapy of arthritic pain using celecoxib, comprising:
a. orally administering a first celecoxib formulation comprising 100 mg of celecoxib to a patient suffering from arthritic pain;
b. determining the celecoxib concentration in the patient's blood at a plurality of time points after the first celecoxib formulation was administered to the patient;
c. transforming the patient's celecoxib concentration/time data points in to one or more pharmacokinetic (PK) parameters;
d. determining the patient's clinical response to the first celecoxib formulation comprising 100 mg of celecoxib and
i. if pain control is not adequate repeat steps a-c with a higher celecoxib dose;
ii if toxicity is not tolerable repeat steps a-c with a lower celecoxib dose;
iii. if pain control is adequate and toxicity is tolerable determine the then present value for one or more pharmacokinetic (PK) parameters; and
e. maintaining the patient on the celecoxib formulation at a celecoxib dose and frequency of administration that results in the value for the one or more pharmacokinetic (PK) parameters determined in step d (iii).

2.-7. (canceled)

8. The method of claim 1, wherein the PK parameter used is one or more of concentration, concentration time course, peak concentration, time after administration to peak concentration, terminal half-life, AUC, bioavailability, absorption, distribution, metabolism, excretion, biotransformation, or a combination thereof.

9. The method of claim 1, further comprising administering pharmaceutically active doses of one or more pharmaceutically active agents in addition to celecoxib.

10. The method of claim 9, wherein the additional pharmaceutically active agents are active agents used to treat central and peripheral nervous system disorders, cardiac arrhythmias, hyperlipidemia, hypertensive, angina, congestive heart failure, respiratory system disorders, gastrointestinal disorders, renal disorders, epilepsy, migraine, sleep disorders, anxiety, substance abuse, pain, parkinsonism, spasticity, psychiatric disorders, dementia, endocrine disorders, diabetes, thyroid disorders, gynecologic and obstetric conditions, lactation, urologic system disorders, immunologic disorders, bone and joint disorders, osteoporosis and osteomalacia, inflammatory disorders, disorders of the eyes, ears, nose, and throat, dermatologic system disorders, hematologic disorders, coagulation disorders, infectious diseases, neoplastic diseases, nutritional disorders, or allergic disorders.

11. The method of claim 10, wherein the celecoxib and the one or more pharmaceutically active agents in addition to said celecoxib are administered in a fixed dose combination.

12. The method of claim 11, wherein the pharmaceutically active agent in addition to celecoxib is hydrochlorothiazide.
